(19) United States
(12) Patent Application Publication
(10) Pub. No.: US 2013/0004546 A1
(43) Pub. Date: Jan. 3, 2013

(54) HYDROGEL OF MICROSPHERES

(75) Inventor: Elizabeth Laugier, Geneve (CH)

(73) Assignee: BIOPHARMEX HOLDING LIMITED, Wanchai (CN)

(21) Appl. No.: 13/522,773

(22) PCT Filed: Jan. 20, 2011
(86) PCT No.: PCT/EP2011/050727

§ 371 Date: Aug. 30, 2012

Related U.S. Application Data
(60) Provisional application No. 61/303,094, filed on Feb. 10, 2010.

(30) Foreign Application Priority Data
Jan. 20, 2010 (FR) 1050355

(51) Int. Cl. A61K 9/10; A61P 19/04; A61P 21/00; A61K 31/717; A61P 17/00

(52) U.S. Cl. 424/400; 514/57

(57) ABSTRACT

The invention relates to an injectable composition intended for tissue induction of the soft tissues by a system of immediate and prolonged progressive release.

In particular, the invention relates to a composition comprising a gel of at least one first biodegradable polymer forming a first agent for filling soft tissues in an animal, and a suspension in this gel of microspheres with an average diameter between 5 and 50 micrometres, said microspheres comprising 50 to 90 wt. % of at least one biodegradable polysaccharide relative to the total weight of the microspheres, said microspheres inducing a second filling by tissue induction and containing at least one second biodegradable polymer forming at least one third filling of the soft tissues in an animal.

The invention relates to an injectable composition intended for tissue induction of soft tissues by a system for immediate and prolonged progressive release.

### PRIOR ART

For decades, a large number of products have been used for inducing neocollagenesis in order to increase the thickness of a soft tissue, by the injection or placement of implants inducing a foreign body reaction described in the prior art (Woodward, 1999; Curtis et al., 1990; Salthouse, 1984; Cotran et al., 1994; Tizard, 1996; Anderson 1994a).

Each of these products has advantages and disadvantages. Essentially there are two categories of products:

### Non-Degradable Products:

The earliest product, silicone gel, is inexpensive but is now prohibited because of certain delayed chronic inflammatory reactions, occurring more than 20 years after injection. As fibrosis was slight initially, the injections had to be repeated and hard masses having a tendency to gravitate could appear during aging of the tissue, giving unattractive results in the long term, a swollen and hard appearance of the tissues, and this product cannot be resorbed.

Teflon caused infections and gross inflammatory reactions and has also been prohibited.

Microspheres of PMMA in a gel of gelatin or of collagen, permitting neocollagenesis of the tissue, represent an advance in the durability of the results, but collagen gel is likely to trigger allergic inflammatory reactions (obligatory test) and the non-resorbable particles have caused tissues to produce fibrous capsules (tendency of tissues to isolate a foreign body) and, through their non-resorbability, also to induce delayed inflammatory reactions, which although rare, are impossible to control because of the non-resorbability of the particles.

### Degradable Products:

Implant based on polylactic acid (PLA) constituted of a carmellose gel containing microspheres of PLA from 40 to 60 microns. A major advantage for lipoatrophies, where fatty tissue is nonexistent and where the fibrosis induced makes it possible to replace the empty spaces with a long-lasting increase in dermis. A drawback is that it is extremely difficult to inject and the PLA particles are resorbed too slowly (more than 3 years), allowing the possibility of creating an inflammatory reaction in the long term and necessitating injections in the deep tissue, so that it is not possible to treat superficial defects.

Hyaluronic acid gel, containing microspheres of calcium triphosphate, also leads to a foreign body reaction, but the particles are long-lasting and can also result in thick fibrous capsules and can cause uncontrolled inflammatory reactions over time.

A patent application WO/2001/012247 of Bioform Laboratories describes a polysaccharide gel selected from the group comprising celluloses containing multiple ceramic particles from 20 to 200 microns for tissue augmentation. The particles are long-lasting (up to 7 years) and they too can lead to uncontrolled inflammatory reactions of the tissues of the inflammatory nodule type called granulomas, which cannot be controlled, having both aesthetic and psychological repercussions.

There is a patent application WO 2008/147817 that describes microcapsules containing hyaluronic acid covered with a layer of polymer, protein or polysaccharide, for protecting the hyaluronic acid during injection and having a delayed effect in release of the hyaluronic acid. However, the particles described for supplying sufficient hyaluronic acid for a filling effect are of large diameter, up to 2 mm. The coated capsules contain the active principle, which will be released completely during membrane resorption, for a varying time depending on the material selected (from a few days to several years).

This encapsulation system employs coating in the form of a membrane for protecting the active substance, it is a reservoir system, and in general the substance can only be released on degradation of the membrane, and it is therefore a delayed release mechanism.

Patent application EP 0 499 164 describes a composition comprising a suspension of a fine powder of a hydrophilic polymer in a liquid carrier constituted of a hydrophobic polymer. The liquid carrier is a hydrophobic polymer that is necessary according to this document for maintaining a suspension of the particles of polymer. In particular, these hydrophobic polymers are, according to the examples, alcohols, glycerol or its derivatives, or a carboxylic acid. This composition is particularly suitable for replacing a lubricating fluid of the human or animal body. Thus, the aim is different from that of the present invention, and the particles are non-degradable particles of methacrylate.

Patent application WO 2009/100422 describes an injectable composition comprising a matrix of a biodegradable gel and a system of microparticles containing a bioactive agent. However, on the one hand the hydrogel envisages the presence of polyethylene glycol, but the microparticles are also prepared from polymers of the polyester type, etc., and in particular of the PLGA type. Thus, this injectable system relates to the prior art and is not particularly suitable for the aims of the present invention.

Finally, patent application WO 2005/105167 describes an injectable composition comprising microparticles prepared from a solution of crosslinked alginate in a bath of barium chloride. However, the microparticles, notably with a size of about 200 microns and constituted solely of crosslinked alginate, are not particularly suitable for the aims of the present invention.

In other more remote technical fields, numerous materials, especially polymers, have been investigated for designing systems with controlled release. These include polyesters, polyanhydrides, PEO, polyamides and several polysaccharides. The interest in biodegradable polymers for sustained release of an active principle began in the 1970s, notably with the work of Yoller et al., polymer news 1:9-15 (1970), and since then numerous polymers have been prepared for the release of active agents. These materials must not contain impurities (solvents) or residues (monomers) from their manufacture. The degradation products must not display any toxicity.

### AIMS OF THE INVENTION

The main aim of the invention is to solve the technical problem consisting of supplying an injectable composition for the augmentation or filling of soft tissues.

In particular the present invention has the aim of supplying an injectable composition for use in the field of plastic surgery, and notably cosmetic surgery, notably for the reconstruction or filling of soft tissues of the human body, the restoration of tissue loss due to accidents or drug-induced (lipoatrophies), as well as those associated with aging (thinning of the dermis and loss of adipose tissue), dental care such as for filling periodontal pockets, or surgery of the vocal cords or management of urinary incontinence.

One of the aims of the invention is to rectify the drawbacks of the known products and improve the performance of the treatments for filling the soft tissues of the face and of the body both quantitatively, in particular by the duration of their action, as the main problem of degradable products for the filling of tissues is their short useful life; and qualitatively by a mechanism of gentle and controlled collagenesis that induces only a very slight, if any, uncontrollable inflammatory risk over the long term, while providing filling for many months without returning to the previous state even after disappearance of said composition. The invention therefore has the aim of supplying an injectable composition for the filling of soft tissues with optimum biodegradation for stimulating the mechanism of collagenesis while avoiding an inflammatory response.

Advantageously, the invention has the aim of supplying an injectable composition permitting, at one and the same time, induced neocollagenesis combined with simultaneous hydration of the soft tissues. Preferably it is not a simple delayed filling but 2 simultaneous effects: the formation of new tissue and hydration of this new tissue at the same time, following placement of the injectable composition.

The invention also has the aim of limiting the number of interventions, in other words limiting the number of injections in a given period of time.

### DESCRIPTION OF THE INVENTION

Thus, the present invention describes a composition comprising a gel of at least one first biodegradable polymer forming a first agent for filling soft tissues in an animal, and a suspension of microspheres with an average diameter between 5 and 50 micrometres in said gel, said microspheres comprising 50 to 90 wt. % of at least one biodegradable polysaccharide relative to the total weight of the microspheres, said microspheres containing at least one second biodegradable polymer forming a second agent for filling soft tissues in an animal.

Said biodegradable polysaccharide advantageously forms a matrix substrate for the microspheres.

It was discovered that said composition makes it possible to solve all of the technical problems mentioned above, reliably, reproducibly, and for use on an industrial scale. The invention can be used in particular in the field of reconstruction of soft tissues, notably of the face and of the human body, for example following accidental tissue loss, or tissue loss associated with a disease (lipoatrophies), or with aging, in the filling of periodontal pockets (alveolodental pyorrhoea), in the treatment of urinary incontinence, or in the surgical treatment of vocal cords.

“Gel of at least one biodegradable polymer” means a gel of at least one polymer that is degraded progressively, and typically over several days but in a period of less than about 6 months, to components of low molecular weight that are eliminated by natural processes when it is injected subcutaneously or intradermally, in the body of a hot-blooded animal, and typically of a human being. A gel is generally defined as a three-dimensional network of solids diluted in a fluid. It is called a hydrogel when the fluid is water. The gel of the invention is typically constituted of an agent for filling soft tissues, and for example alginate, or glycosaminoglycans, such as chondroitin sulphate, dermatan sulphate, keratan sulphate, heparin or heparan sulphate, and preferably hyaluronic acid. The invention also covers gels based on mixtures of alginate and/or of glycosaminoglycans, and preferably of hyaluronic acid. Agents for filling soft tissues are known by a person skilled in the art of plastic surgery, and some are referred to in the work: “Esthétique du visage: Techniques de comblement et remodelage, Avancées en dermatologie cosmétique” (Aesthetics of the face: Techniques for filling and remodeling, Advances in cosmetic dermatology), Jean Carruthers, Daphné Thioly-Bensoussan, Elsevier Masson, 2005” or in “L'art du comblement et de la volumétrie en esthétique” (The art of filling and of volumetry in cosmetic surgery), Dr Annick Pons Guiraud, 1999”.

“Microspheres” are, within the scope of the invention, matrices comprising one or more biodegradable polysaccharides. This term does not cover microcapsules, which envisage coating of the gel with a protective layer of the “coating” type.

The microsphere or dispersed matrix system is a matrix of polymer with the filling agent or agents dispersed in said matrix, release is immediate following placement of the system, it is progressive, and continues up to complete resorption of the matrix. It is therefore an immediate, progressive and controlled release, depending on the rate of resorption of said matrix in the living environment. The dispersed matrix system or microsphere of the invention therefore delivers the filling agent by progressive diffusion following placement in the tissue, the diffusion of said filling agent being concomitant with the resorption of the matrix for a time that is defined and known.

The combination of a biodegradable polysaccharide and a biodegradable polymeric filling agent makes it possible to form microspheres that are particularly suitable for solving all of the technical problems that need to be solved.

Furthermore, the microspheres of the present invention make it possible to induce a second filling by tissue induction, beyond the first effect of physical filling.

The proportion of 50 to 90 wt. % of at least one biodegradable polysaccharide relative to the total weight of the microspheres refers to the biodegradable polysaccharide to the exclusion of the filling agent or agents contained in the microspheres, which can also be polysaccharides (for example: hyaluronate).

Advantageously, the microspheres are bioresorbable. “Bioresorbable” means microspheres that are resorbed naturally, and preferably whose metabolites, which are nontoxic, are eliminated by the body's natural processes, preferably between about one and six months of being in the body of a hot-blooded animal, and typically a human being.

The polymers of the invention (the polysaccharide and the biodegradable polymers) are not crosslinked but are rather kept preferably as native polymers, i.e. without further crosslinking step. A further crosslinking of the polymers is preferably avoided not to prepare sustained release polymers which have different properties notably in term of time life when used in vivo than the polymers of the invention. The polymers of the invention are preferably biopolymers, i.e. already existing in nature, either prepared by isolation from products of nature or by synthesis.

Thus, according to the invention, the microspheres are in suspension in a gel of at least one biodegradable polymer forming a filling agent. This polymer gel can be formed from a polymer or mixture of polymers identical to those enclosed in the microspheres. Advantageously this makes it possible to supplement the filling effect or action of the polymer gel by supplying a filling agent released by the microspheres.

In particular, the microspheres are prepared by spraying.

Advantageously, the microspheres have an average diameter between 5 and 50 microns, and preferably between 10 and 40 microns, and more preferably between 20 and 40 microns.

Typically, the apparent density of the microspheres is between 100 and 800 g/L.

According to a preferred embodiment, said first and second agents for filling soft tissues in an animal are a glycosaminoglycan, identical or different, and are preferably hyaluronic acid or a hyaluronate, or a salt thereof.

According to a preferred embodiment, the biodegradable polysaccharide used in the preparation of the microspheres is selected from the group comprising a derivative of cellulose, a cellulose ether, for example a carboxyalkyl cellulose, and preferably carboxymethyl cellulose, an alkylhydroxy cellulose, and preferably ethylhydroxymethyl cellulose, ethylmethyl cellulose, hydroxypropyl methyl cellulose, hydroxymethyl cellulose, hydroxypropyl cellulose, hydroxymethylpropyl cellulose, an alkyl cellulose, and preferably an ethyl cellulose or a methyl cellulose, or a cellulose ester, for example a cellulose acetate, a cellulose propionate; a cellulose nitrate; a gum such as gum arabic, a maltodextrin, a starch, a derivative of starch (for example selected according to The International Numbering System for Food Additives), a chitosan, a chitin, an agarose, a trehalose and any mixture thereof.

Preferably, microspheres comprise or are constituted of a mixture in matrix form of carboxymethyl cellulose and of calcium alginate, or of carboxymethyl cellulose, of alginate, and of hyaluronic acid, or a salt thereof, or of carboxymethyl cellulose, of alginate, of hyaluronic acid, or a salt thereof, and of starch, and/or of maltodextrin, and/or of trehalose.

Among the salts of hyaluronic acid, reference is made to the hyaluronates, notably in the form of salt of sodium, of calcium, of barium or of potassium.

Among the salts of alginate, reference is made in particular to the salts of calcium or of sodium.

Advantageously, the biodegradable polymers are of vegetable or synthetic origin, notably to remove any risk of a possible allergy to animal derivatives.

According to a preferred embodiment, the microspheres comprise or are constituted of a mixture in matrix form of cellulose derivatives, preferably of carboxymethyl cellulose and hydroxypropyl cellulose; and/or of a derivative of starch and/or of maltodextrin and/or of trehalose; and of a component selected from the glycosaminoglycan family, for example a hyaluronate derivative (sodium salt, calcium salt, barium salt or potassium salt) and/or an alginate derivative (calcium salt or sodium salt).

Advantageously, the microspheres comprise, as biodegradable polysaccharide, a mixture of at least two cellulose derivatives, and preferably carboxymethyl cellulose (CMC) and hydroxypropyl cellulose (HPC).

Preferably, the suspension is sterile. A typical stage of sterilization is typically a back-pressure autoclave treatment.

The gel carrying the particles of the invention is typically a hydrogel of alginate or a salt thereof, preferably calcium alginate or sodium alginate, or a hydrogel of hyaluronic acid or a salt thereof, and preferably a calcium hyaluronate or sodium hyaluronate.

According to one embodiment the carrying gel is a biodegradable hydrogel comprising or composed of at least 2 polymers, one of which is selected from the glycosaminoglycan family, and preferably a sodium hyaluronate, and another is selected from a cellulose derivative, and preferably carboxymethyl cellulose. The presence of a cellulose derivative makes it possible to improve the stability of the suspension.

According to a preferred variant, the composition of the invention comprises a hydrogel of alginate or a salt thereof, preferably calcium alginate or sodium alginate, and a suspension of microspheres of at least one polysaccharide containing a hydrogel of alginate or a salt thereof, preferably calcium alginate or sodium alginate.

According to a preferred variant, the composition of the invention comprises a gel of hyaluronic acid, or a salt thereof, and optionally a cellulose derivative, and preferably carboxymethyl cellulose, and a suspension of microspheres of at least one polysaccharide containing a gel of hyaluronic acid, or a salt thereof, preferably calcium hyaluronate or sodium hyaluronate.

According to a preferred variant, the composition of the invention comprises a gel of hyaluronic acid, or a salt thereof, and optionally a cellulose derivative, and preferably carboxymethyl cellulose, and a suspension of microspheres comprising 3 components (two polymers forming filling agents and a polysaccharide), and preferably a mixture of hyaluronate, alginate, and cellulose, or their derivatives.

According to a preferred variant, the composition of the invention comprises a gel of hyaluronic acid, or a salt thereof and optionally a cellulose derivative, and preferably carboxymethyl cellulose, and a suspension of microspheres comprising 4 components (two polymers forming filling agents and two polysaccharides), and preferably a mixture of hyaluronate, alginate, cellulose, and starch, or their derivatives.

According to a preferred variant, the composition of the invention comprises a gel of hyaluronic acid, or a salt thereof and optionally a cellulose derivative, and preferably carboxymethyl cellulose, and a suspension of microspheres comprising 5 components (two polymers forming filling agents and three polysaccharides), and preferably a mixture of hyaluronate, alginate, cellulose, agarose, maltodextrin, or trehalose.

Advantageously the microspheres of the invention comprise, as biodegradable polysaccharide, a mixture of at least two derivatives of cellulose, and preferably of carboxymethyl cellulose (CMC) and hydroxypropyl cellulose (HPC).

The present invention also relates to an injectable implant for the filling of the soft tissues, characterized in that it comprises or is constituted of a composition as defined previously.

Advantageously, the present invention also relates to an injectable implant for the hydration of soft tissues, and in particular of filled tissues, characterized in that it comprises or is constituted of a composition as defined previously.

According to one variant the implant is injectable by subcutaneous, intradermal, or intramuscular, or intracartilaginous, or intramucosal route.

According to a particular embodiment, the implant is intended for reconstructive or cosmetic surgery of soft tissues, in particular of the face and of the body, notably within the scope of tissue losses associated with an accident, with aging, or secondary to certain diseases such as an immunodeficiency, such as for HIV patients, and in particular lipoatrophy, with a periodontal disease (periodontal pocket), or the treatment of urinary incontinence, or surgery of vocal cords.

The present invention notably makes it possible to obtain a stimulatory effect (tissue induction, i.e.

inducing the formation of tissue) simultaneously with creation of new tissue and local hydration.

The present invention notably makes it possible to set up 3 different mechanisms in a single application. A first mechanism of filling associated with the carrying gel, simple and immediate, a second mechanism of filling in the subsequent 1 to 6 months by the foreign body reaction (due to the microspheres) leading to controlled neocollagenesis, and therefore to durability of said primer filling, and a third mechanism of direct hydration by the progressive and continuous diffusion of the gel in the tissues following degradation of the particles, improving the quality of the tissue in situ and of the new tissue without additional intervention.

Thus, the invention relates to a composition intended for a triple action in the region of tissue injected: direct effect of filling of the tissue, tissue induction effect by neocollagenesis with lifting of the tissue for about 45 days, and tissue filling by progressive salting out of the filling agent or agents following degradation of the microspheres.

On placement of an implant, the inflammatory reaction is basically a protective response with the aim of removing the foreign body from the site of implantation.

The first phase, of short duration, is at first triggered by the action itself (surgery or injection).

The second phase is a vascular phase: increase in blood flow and vascular permeability to allow the plasma proteins and leukocytes to leave the bloodstream and go to the site of implantation, and neutrophils and monocytes to clean the site of the foreign body.

The third phase corresponds to phagocytosis, recognition of the implant or implants to be ingested and complete degradation if the material is biodegradable. There is release of enzymes (collagenases and elastases), influx of fibroblasts activated by growth factors and activation of these fibroblasts for tissue repair leading to regeneration of the tissue in situ by remodeling of the connective tissue, manifested by synthesis of collagen.

If the surface area of the implant is too large there is increase of the inflammatory reaction, causing cellular damage, which is the reason why the implant selected contains matrices preferably with average diameter of 30 to 40 microns.

On the other hand an implant that is non-degradable or slowly degradable leads to the formation of a fibrous capsule isolating the foreign body, which can give rise to severe reactions that do not come to an end, since the foreign body cannot be digested by said cells. That is why quickly degradable polymers are chosen, which trigger collagenesis but lead to hardly any possibility of permanent inflammatory reaction that could create defects that are unsightly, troublesome or non-functional, on the surface of the soft tissue.

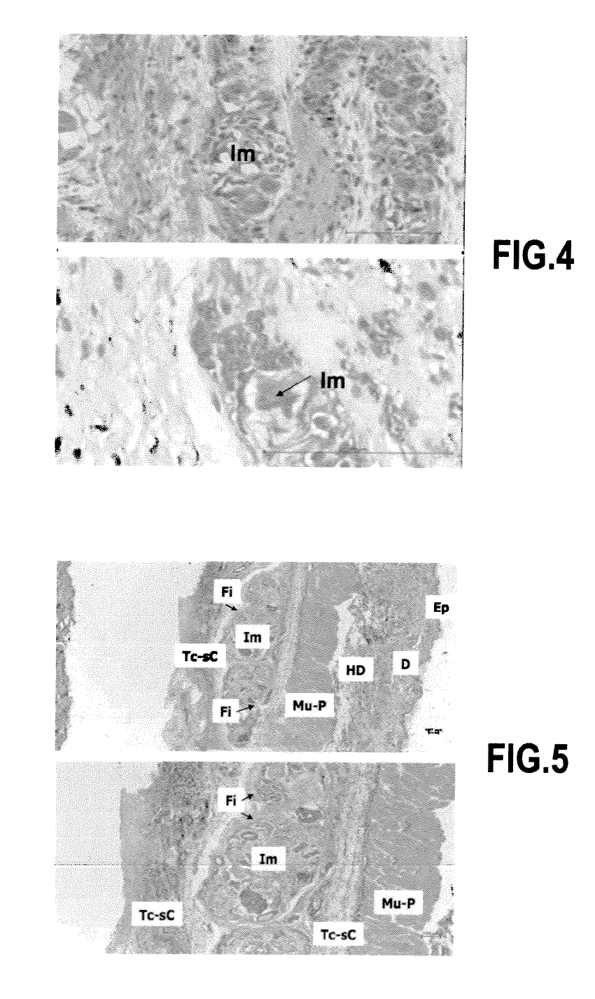

The implant of the invention, once put in place, will degrade, releasing the various polymers making up the matrix of the microspheres, while inducing the required tissue lifting reaction. This is confirmed by the photographs of the tissues (FIGS. 4 and 5).

When making this choice, for this tissue reaction to take place it is important to have an implant that is rapidly resorbable but degradable in not less than about 1 month to 2 months to give time for development of the mechanism of neocollagenesis. More than about 6 months does not prove necessary, as the collagenesis developing in the initial weeks stops anyway, even if the implant persists, but the inflammatory reaction and the creation of fibrous capsules will not stop, which will become the drawbacks of this chosen therapeutic method. It is then better to have a second stimulation with this same implant at about 3 months for a new lifting of the tissue, as then the tissue will be completely or almost free of the previous placement of the matrices, which will have been phagocytosed.

Ultrasound scans carried out successively at 15 days and then at 30 days clearly show that the lifting of the tissue (about 1 to 2 mn depending on the subject's age and the site injected in 1 single placement of the implant) occurs early and is long-lasting (at least 4 months) while allowing the injected tissue to recover its integrity. The tissue reaction of lifting of the tissue stops anyway after 2 months even if the implant is still in place.

FIGS. 4 and 5 show the tissues after injecting the implant.

Gradual supply of the combination of various polymers forming the matrix to the tissue during its degradation additionally provides simultaneous hydration of the new tissue owing to the physicochemical properties of the polymers used and selected and which are known to have strong hydrating power.

Thus, preferred filling agents are those that hydrate newly synthesized tissues.

In particular, hyaluronic acid is known for its role in hydration, acting by its interactions with binding proteins and especially with its transmembrane receptor (Tool B. P. 2001, sem. cell. Devel. Biol.12: 79-87, Stuart A. J., Drug Delivery, 12: 327-342, 2005). Activation of this receptor plays a role in cell multiplication and proliferation, and in angiogenesis. Therefore this tertiary action (action 1 gives a simple volume connected with the carrier gel, action 2 gives collagenesis connected with reaction of the tissue where the matrices were placed) makes it possible to maintain the volume and hydration of the skin, a true bath for the tissue to be regenerated, permitting an average duration of 3 to 6 months.

Similarly alginic acid or its salts is a polysaccharide that possesses properties of swelling and hydration, which are extremely useful in tissue reconstruction.

These polymers are therefore polymers representing filling agents of choice for the present invention.

The microspheres of the invention have a useful life of to 6 months with a very moderate inflammatory response, preventing any delayed reaction, with the phenomenon of granulation stopping on disappearance of the microspheres and permitting a new supply of the gel or gels of biodegradable polymer progressively released by replacing the initial carrying gel, further increasing the results of filling, and improving the duration of filling as well as the quality of the tissue.

In fact, an implant is a foreign body, it is not desirable for its life to exceed 6 months since the reaction of collagenesis is rapid between 30 and 60 days for one application of the product and serves as a mechanical filling that is longer-lasting than the products based on unencapsulated native hyaluronate or native alginate.

The present invention permits the filling of tissues, notably owing to the tolerance, the durability (neither too long nor too short) and to the body's tolerance to the products used, or even the elimination of long-term risks, while maintaining convenience of use, good injectability, and a resorption time that is well defined, whether it is that of the carrier (gel) or that of the microspheres.

Advantageously, the present invention makes it possible to avoid the tests of allergenicity.

The invention also relates to a kit for reconstructive or cosmetic surgery comprising an injectable implant as defined previously and an injection syringe, preferably ready to use.

Advantageously, the injection syringe includes a needle having a gauge between 26 G and 30 G. The composition of the invention is particularly suitable for said needles.

The invention relates to a method for cosmetic dermatologic care, characterized in that it comprises the injection of one or more doses of a composition as defined previously or of an injectable implant as defined previously.

In particular, the method of cosmetic dermatologic care is intended for reconstructive or cosmetic surgery of the soft tissues, in particular of the face and of the body, notably within the scope of tissue losses in connection with an accident, with aging, or secondary to certain diseases such as an immunodeficiency, and in particular lipoatrophy, a periodontal disease (periodontal pocket), the treatment of urinary incontinence, or surgery of the vocal cords.

A method of cosmetic dermatology has the aim of improving the patient's appearance.

Advantageously, the method of cosmetic dermatologic care of the invention is intended for dermal filling, and/or subcutaneous filling, and/or filling of muscle, and/or of cartilage, and/or of the mucosae.

The microspheres of the invention do not dissolve in the gel. The microspheres are fairly resistant to the mechanical stresses associated with suspending them in the gel. The microspheres are also fairly stable (significant absence of release of the filling agent contained in the microspheres) during sterilization at 120° C. for 20 to 40 minutes, and do not dissolve in the hydrogel over time (about 2 years) at room temperature after sterilization.

On the drawings:

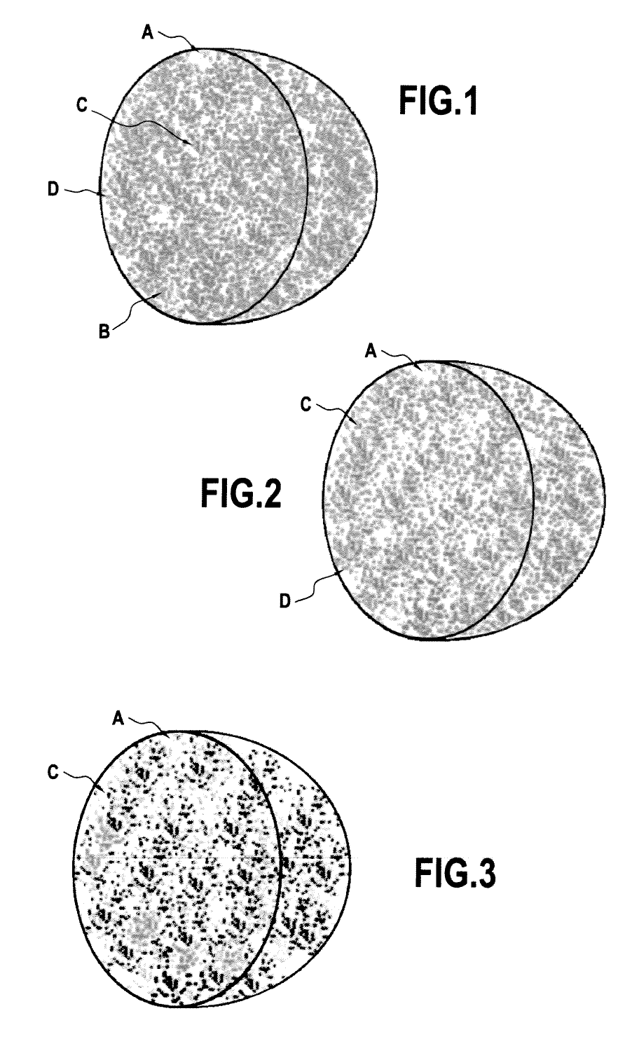

FIG. 1 is a schematic representation of a microsphere of the invention comprising a matrix of four biodegradable polysaccharides, two of which are filling agents (HA and alginate);

FIG. 2 is a schematic representation of a microsphere of the invention comprising a matrix of three biodegradable polysaccharides, two of which are filling agents;

FIG. 3 is a schematic representation of a microsphere of the invention comprising a matrix of two biodegradable polysaccharides, one of which is a filling agent;

In FIGS. 1, 2 and 3:

A=carboxymethyl cellulose (CMC)

B=starch

C=hyaluronic acid (HA)

D=alginate

FIG. 4 is a photograph of a histological section of a tissue after subcutaneous injection of 0.2 mL of a composition comprising a suspension of microspheres of CMC and native hyaluronic acid, dispersed in a hydrogel of CMC at 0.5%.

FIG. 5 is a photograph of subcutaneous injection of a composition of the invention comprising 0.2 mL of a suspension of microspheres of CMC, with a native hyaluronic acid and a calcium alginate, dispersed in a hydrogel of CMC at 0.5%.

In the hydrogel used, CMC (carboxymethyl cellulose) was used instead of a filling agent such as hyaluronic acid to investigate the biodegradation of the microspheres alone.

Legend: Ep: Epidermis, D: Dermis, HD: hypodermis, mu-P: cutaneous muscle.

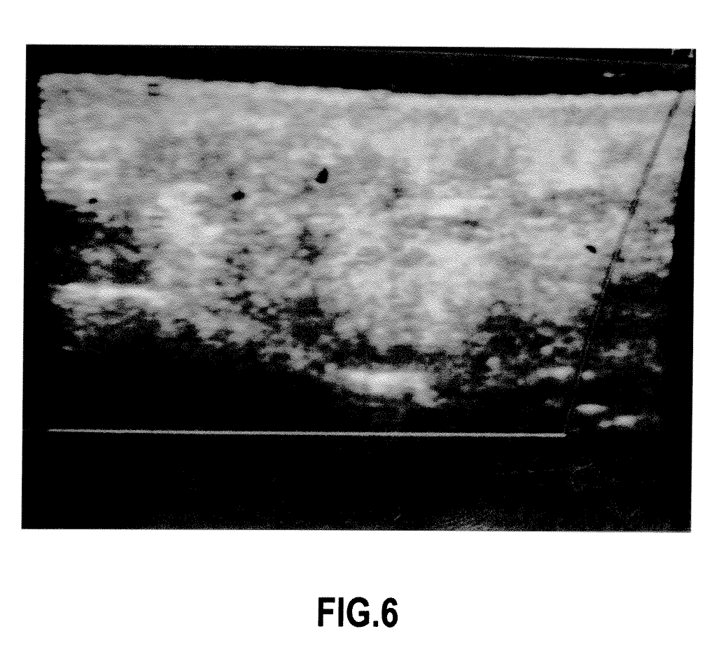

FIG. 6 shows an ultrasound scan carried out 7 weeks after injection of a composition of the invention.

The compounds according to the present invention are prepared in the form of topical compositions, notably cosmetic dermatological compositions, for cosmetic dermatology.

The term “effective amounts”, as used here, means an amount of a compound or composition sufficient for significantly inducing a positive effect, including—independently or in combination—the effects described in the present invention.

Other aims, characteristics and advantages of the invention will become clear to a person skilled in the art on reading the explanatory description, which makes reference to examples that are only given for purposes of illustration and are not intended to limit the scope of the invention in any way.

The examples form an integral part of the present invention and any characteristic that is novel relative to any prior art from the description taken in its entirety, including the examples, forms an integral part of the invention in its function and in its generality.

Thus, each example is of a general scope.

Moreover, in the examples, all the percentages are percentages by weight, unless stated otherwise, and the temperature is expressed in degrees Celsius unless stated otherwise, and pressure is atmospheric pressure, unless stated otherwise.

### EXAMPLES

### Example 1

### Preparation of a Biodegradable Polymer Gel

A solution of a carrier fluid or hydrogel for suspending the microspheres (for example a solution or a gel of hyaluronic acid with a molecular weight of 2 million dalton with a concentration between 0.5 and 3 vol. %) is prepared as follows:

Put 6 g of sodium chloride in 600 ml of sterile water in a 1 L glass reactor.

After dissolving the sodium chloride with a sonicator, add 4 g of hyaluronic acid (MW 2 million dalton), carefully unraveling the fibers of hyaluronic acid by hand as far as possible.

After stirring the heterogeneous mixture with a spatula for 2 minutes, leave the reactor at 4° C. for 20 h without stirring, covered with aluminum foil to protect the reaction mixture.

### Example 2

### Preparation of Microspheres of the Invention

### Production of the Microspheres of the Invention by Spraying a Solution and Final Chemical Reaction:

A compound of gel of sodium hyaluronate is prepared in order to obtain resistance to heat, by combining a starch gel and a dextran gel with a gel of CMC, to form matrices, said matrices having a size of 20 to 40 microns.

The microspheres are prepared independently of the hydrogel.

### Method:

preparation of the spraying solution:

dissolve 64 g of carboxymethylcellulose in 800 ml of previously heated (50+/−5° C.) purified water, with stirring;

add 200 ml of isopropanol, then 8 g of sodium hyaluronate, 4 g of maltodextrin, and 4 g of native starch gel;

continue stirring the suspension with a magnetic bar

adjustment of the sprayer to obtain microspheres of 30 microns (SD 10 microns):

use a sprayer of the BUCHI B-290 type;

set the compressed air at 6 bar;

air/solvent aspirating pump at 100%;

peristaltic pump for withdrawing the suspension set at 30%;

injection temperature 180° C.;

the outlet temperature will be about 80° C.;

automatic cleaning of the injection nozzle set at 2s (scale from 0 to 9);

spraying:

prior calibration of the apparatus with rinsing water;

adjustment of the injection temperature to obtain an outlet temperature of 80° C.;

starting the treatment;

recovery of the matrices in the form of microspheres;

leave the machine to cool down;

remove the cyclone and recover the powder (about 80 g);

final chemical reaction:

T zero: in a 500 ml beaker, pour 400 ml of isopropanol+50 g of above manufactured microspheres; stir;

T 10 min: add calcium chloride in stoichiometric quantity, stir for 30 min;

T 40 min: after 30 minutes, add 50 ml of purified water, stirring vigorously;

T 50 min: after 10 minutes, put in a stove at 105° C. for drying;

Recovery of the matrices in the form of microspheres.

### Example 3

### Preparation of Microspheres of the Invention

### Production of the Microspheres by Direct Spraying:

A compound of gel of alginate and calcium hyaluronate is prepared in order to obtain resistance to heat, by combining with a gel of CMC to form matrices, said matrices having a size of 20 to 40 microns.

The microspheres are prepared independently of the biodegradable polymer gel.

### Method:

preparation of the spraying solution:

dissolve 40 g of carboxymethylcellulose and 24 g of HPC in 600 ml of previously heated (60+/−5° C.) purified water, with stirring;

add 400 ml of isopropanol, then 8 g of calcium alginate and 8 g of calcium hyaluronate;

continue stirring the suspension with a magnetic bar;

adjustment of the sprayer to obtain matrices in the form of microspheres of 30 microns (SD 10 microns):

use a sprayer of the BUCHI B-290 type;

set the compressed air at 6 bar;

air/solvent aspirating pump at 100%;

peristaltic pump for withdrawing the suspension set at 30%;

injection temperature 180° C.;

the outlet temperature will be about 80° C.;

automatic cleaning of the injection nozzle set 7s (scale from 0 to 9):

spraying;

prior calibration of the apparatus with rinsing water;

adjustment of the injection temperature to obtain an outlet temperature of 80° C.;

starting the treatment;

recovery of the matrices in the form of microspheres:

leave the machine to cool down;

remove the cyclone and recover the powder (about 80 g);

### Example 4

### Preparation of a Biopolymer Gel

A solution of a hydrogel (for example a solution or a gel of hyaluronic acid with molecular weight of 2.5 million dalton with a concentration between 1 and 2 vol. %) is prepared as follows:

Put 6 g of sodium chloride in 600 ml of sterile water in a 1 L glass reactor.

After dissolving the sodium chloride with a sonicator, add 4 g of hyaluronic acid (MW 2.5 million dalton), carefully unraveling the fibers of hyaluronic acid by hand as far as possible.

After stirring the heterogeneous mixture with a spatula for 2 minutes, leave the reactor at 4° C. for 20 h without stirring, covered with aluminum foil to protect the reaction mixture.

### Example 5

### Suspending the Microspheres in the Biodegradable Polymer Gel:

Then put 40, 30 or 20 vol. % of dispersed matrix phase (microspheres) from example 2 or 3 in the gel from example 1 or 4, so as to obtain a homogeneous suspension, typically by using a homogenization means of the mixer type.

The injectable implant according to the invention can be presented in the form of a ready-to-use prefilled syringe, a ready-to-use prefilled bottle, or a lyophilizate to be reconstituted as and when required.

### Example 6

### Characterization of the Microspheres of the Invention

Table 1 shows some characteristics of the microspheres of the invention. Note that the size or average diameter of the microspheres can be adjusted depending on the proportions of the various constituents.

TABLE 1

| | Composition No. (microspheres) | | | | | | | | | | | | |
|---|---|---|---|---|---|---|---|---|---|---|---|---|---|
| | 1 | | 2 | | 3 | 4 | | 5 | | 6 | | 7 | |
| Ratio | 20%/ | 40%/ | 20%/ | 40%/ | 40%/ | 20%/ | 40%/ | 20%/ | 40%/ | 20%/ | 40%/ | 20%/ | 40%/ |
| Hyaluronate/ | 80% | 60% | 80% | 60% | 60% | 80% | 60% | 80% | 60% | 80% | 60% | 80% | 60% |
| polysaccharide | | | | | | | | | | | | | |
| substrate (w/w) | | | | | | | | | | | | | |
| Polysaccharide | CMC | | CMC + | | Ethyl- | Gum | | Native | | CGM + | | CGM + HPC + | |
| forming substrate | | | HPC | | cellulose | arabic + | | starch + | | maltodextrin + | | maltodextrin + | |
| in the microspheres | | | | | | maltodextrin | | maltodextrin | | gum arabic | | gum arabic | |
| H % | 6.71 | 5.05 | 4.4 | 3.91 | 1.59 | 4.09 | 2.72 | 4.36 | 2.37 | 4.24 | 3.89 | 5.73 | 3.75 |
| Apparent density Do | 542 | 657 | 107 | 210 | 489 | 400 | 518 | 412 | 484 | 550 | 656 | 439 | 400 |
| in g/L | | | | | | | | | | | | | |
| Average diameter of | 13.6 | 13.04 | 29.76 | 21.52 | 96.12 | 19.62 | 18.53 | 15.7 | 17.96 | 22.27 | 15.53 | 20.02 | 18.98 |
| the microspheres | | | | | | | | | | | | | |
| D(v0.5) in microns | | | | | | | | | | | | | |
| (μm) | | | | | | | | | | | | | |

CMC (carboxymethylcellulose) → BLANOSE CMC - FT
HPC (hydroxypropylcellulose) → KLUCEL ELF PHARM -TDS
Ethylcellulose → AQUALON ETHYLCELLULOSE N7 PHARMA - FT
Gum arabic → SPRAYGUM BB - TDS
Maltodextrin → GLUCIDEX 6 - FT
Native starch → Maize starch pharma Maisista 21.003 - FT

H % means “percentage moisture”. This is always measured after lyophilization and spraying. This makes it possible to calculate the residual dry matter of the product, since all the calculations of formulae are carried out for dry matter.

Apparent density: this is “Dzero”. It signifies “density of the powder without tamping”. This is to be compared with “Dten” (burette tapped 10 times to make the powder settle). The principle consists of pouring exactly 250 ml of powder into a calibrated cylinder, then weighing to obtain an apparent density in grams per liter.

The average diameter is measured according to the following protocol: The diameter of the microspheres is measured by means of a laser granulometer, in this case of the MALVERN type, hence the precision of the results given.

Several tests for manufacture of the matrices were carried out by combining 1 or more elements in order to understand and analyse the reaction of the tissue in contact with said microspheres. The diameter of the microspheres and evaluation of their density makes it possible to modify the method of manufacture according to the elements selected for formation of the microspheres. The filling agent (active principle) incorporated in the microspheres was hyaluronate for this test, but the initial principal aim was to test the matrices in a living tissue.

### Example 7

Injection of a Composition of the Invention

### Investigation and Objective Assessment of Tissue Induction (Foreign Body Reaction) by Injection of a Composition of the Invention Comprising 2 or 3 Resorbable Polymers.

The purpose of the study was to evaluate the local effects after implantation in subcutaneous tissues.

In the rat, 13 elements of various compositions and combinations of matrices in a test for visualizing the tissue induction reaction after a period of 4 weeks.

Each test element was injected once in several females at a rate of 2 injections per element; in parallel, the animals received an injection of a control element (CMC at 0.5% or 2%) in the same conditions.

The effects induced by the test elements were compared with those of the control elements on the same animals.

After implantation, the animals were observed daily throughout the period of observation of 4 weeks; at the end of the period of implantation, histological examinations of the skin sites injected were carried out.

The rat is the “rodent” species usually employed and recommended by the official authorities for evaluation of the tolerance of test elements by this type of method.

The methodology employed was adapted from standard NF EN ISO 10993-6—August 2007 relating to “The biological effects of medical devices—Tests relating to local effects after implantation”.

### Test System

Species: Albino rats SPF (specific pathogen free) Sprague-Dawley

Origin: bred JANUARY (53940 Le Genest-St-Isle, France)

Age: about 8 weeks (at the start of the experiments)

Number and sex: 13 females, nulliparous and non-pregnant

Acclimatization: the animals were acclimatized to the experimental conditions for 5 days before implantation.

Weighing: before implantation, the animals were weighed. They were distributed into groups randomly, according to the randomization table of Moses and Oakfoard. For each group, the mean weight was calculated and the acceptable limits were deduced, the extreme individual weights of the animals must not deviate from the mean weight by +/−10%.

Identification: the animals were identified individually per cage, by marking with picric acid with a brush; the location of the marking, different for each animal, corresponded to a number. Additional marking on the right forepaw and tail marking with a red marker pen identified the study.

Housing: the animals were housed at a rate of 3 or 4 per cage, in polypropylene cages of 60 cm×38.5 cm×20 cm equipped with a stainless steel cover until the end of the experimental period.

Implants: (CMC was used instead of a filling agent such as a gel of hyaluronic acid or of alginate to study the biodegradation of the microspheres alone)

### Implant No. 1:

Carrier gel of CMC;

Microsphere:
Native HA 20%;
CMC 80%.

### Implant No. 2;

Carrier gel of CMC

Microsphere:
Native HA 20%;
CMC+HPC (hydroxypropylcellulose) 80%.

### Implantation of the Elements to be Tested

The day before implantation (D−1), all the animals were shaved on the back in 2 zones on either side of the vertebral column.

On the day of the experiment (D1), each weighed animal was anaesthetized with ether and then asepsis of the shaved skin was assured with an antiseptic dermal solution of Povidone® at 10%.

Each test element was implanted in the subcutaneous tissues of the shaved animal. Each element was injected at a rate of 2 injections of 0.2 ml in 2 animals (1 ml syringe, 26 G needle) apart from test elements 1 and 2 in rat 6919 (incision+trocar).

Similarly, the animals received 1 injection of the control element in the subcutaneous tissues in the same conditions.

### Period of Implantation

One period of implantation was carried out: 4 weeks.

The distribution of the animals was as follows:

13 test elements

2 control elements

2×0.2 ml each

1×0.2 ml each for 4 weeks

(rat 1 to rat 13)

Overall, 2 samples of each test element and 1 sample of each control element were tested. No local reaction (redness, oedema) was noted throughout the test.

The results obtained for implant No. 1 can be seen in FIG. 4.

In particular, FIG. 4 (top photograph) shows two small accumulations of implant (Im) surrounded by occasional lymphocytes, plasmocytes and numerous giant cells and macrophages, or their cytoplasms are filled with dense phagocytosed elements, evidence of a foreign body reaction, the first phase of tissue induction connected with the presence of the microspheres.

In the bottom photograph, the small accumulations of implants are surrounded by numerous foamy macrophages filled with phagocytosed elements.

The results obtained for implant No. 2 can be seen in FIG. 5.

FIG. 5 shows in particular an implant (IM) located in the subcutaneous connective tissue (Tc-sC) in the form of violet accumulations surrounded by light bands of fibrosis (Fi), evidence of the tissue elevation secondary to neocollagenesis.

### Example 8

### Confirmation of the Filling Effect

An ultrasound scan (FIG. 6) was conducted on a region of skin where an implant of the invention (implant No. 2) was injected according to example 7.

The ultrasound scan was carried out 7 weeks after injection. Tissue induction is obtained, with filling of more than 2 mm of dermis post-implantation owing to the implant of the invention.

1. Composition comprising a gel of at least one first biodegradable polymer forming a first agent for filling soft tissues in an animal, and a suspension in this gel of microspheres with an average diameter between 5 and micrometres, said microspheres comprising 50 to 90 wt. % of at least one biodegradable polysaccharide relative to the total weight of the microspheres, said microspheres containing at least one second biodegradable polymer forming a second agent for filling soft tissues in an animal.

2. Composition according to claim 1, characterized in that the microspheres have an average diameter between and 40 microns, and preferably between 10 and 40 microns, and more preferably between 20 and 40 microns.

3. Composition according to claim 1 or 2, characterized in that said first and second agents for filling soft tissues in an animal are a glycosaminoglycan, identical or different.

4. Composition according to claim 3, characterized in that said glycosaminoglycan is hyaluronic acid or a hyaluronate, or a salt thereof.

5. Composition according to claim 1 to 4, characterized in that the polysaccharide is selected from the group consisting of a derivative of cellulose, a cellulose ether, for example a carboxyalkyl cellulose, and preferably carboxymethyl cellulose, an alkylhydroxy cellulose, and preferably ethylhydroxymethyl cellulose, ethylmethyl cellulose, hydroxypropyl methyl cellulose, hydroxymethyl cellulose, hydroxypropyl cellulose, hydroxymethylpropyl cellulose, an alkyl cellulose, an ethyl cellulose, or a methyl cellulose; a cellulose ester, for example a cellulose acetate, a cellulose propionate; a cellulose nitrate; a gum such as gum arabic, a maltodextrin, a starch, a derivative of starch, a chitosan, a chitin, an agarose, a trehalose, and any mixture thereof.

6. Composition according to any one of claims 1 to 5, characterized in that the microspheres comprise or are constituted of a mixture in matrix form of cellulose derivatives, preferably of carboxymethyl cellulose and hydroxypropyl cellulose; and/or of a derivative of starch and/or of maltodextrin and/or of trehalose; and of an element selected from the group consisting of the glycosaminoglycan family, for example hyaluronate (salt of sodium, of calcium, of barium or of potassium) or a derivative thereof and/or alginate (calcium salt or sodium salt) or a derivative thereof.

7. Composition according to any one of claims 1 to 6, characterized in that the microspheres comprise, as biodegradable polysaccharide, a mixture of at least two derivatives of cellulose, and preferably carboxymethyl cellulose (CMC) and hydroxypropyl cellulose (HPC).

8. Composition according to any one of claims 1 to 7, characterized in that the carrying gel is a biodegradable hydrogel comprising or composed of at least 2 polymers, one of which is selected from the glycosaminoglycan family, and is preferably a sodium hyaluronate, and the other is selected from cellulose derivatives, and preferably carboxymethyl cellulose.

9. Composition according to any one of claims 1 to 8, characterized in that it is intended for a triple action in the tissue region to be injected: direct filling effect of the tissue, effect of tissue induction by neocollagenesis with lifting of the tissue for about 45 days, and tissue filling by salting out of the filling agent or agents progressively following degradation of the microspheres.

10. Injectable implant for the filling of soft tissues, characterized in that it comprises or is constituted of a composition according to any one of claims 1 to 9.

11. Injectable implant according to claim 8, characterized in that it is injected by subcutaneous, intradermal, intragingival, intraarticular, or intramuscular route.

12. Injectable implant according to claim 10 or 11, characterized in that it is intended for dermo-reconstruction of tissues, for example in case of tissue loss connected with an accident, with aging, or secondary to certain diseases such as an immunodeficiency, such as for HIV patients, and in particular lipoatrophy, with a periodontal disease (periodontal pocket), or the treatment of urinary incontinence, or surgery of vocal cords.

13. Kit for reconstructive or cosmetic surgery comprising an injectable implant according to any one of claims 8 to 10, and an injection syringe.

14. Method of cosmetic dermatologic care, characterized in that said method comprises injecting one or more doses of a composition according to any one of claims 1 to 9 or of an injectable implant according to any one of claims 10 to 12.

15. Method of cosmetic dermatologic care according to claim 14, characterized in that said method is intended for reconstructive or cosmetic surgery of soft tissues, in particular of the face and of the body, notably within the scope of tissue loss connected with an accident, with aging, or secondary to certain diseases such as an immunodeficiency, and in particular lipoatrophy, a periodontal disease (periodontal pocket), the treatment of urinary incontinence, or surgery of vocal cords.

16. Method of cosmetic dermatologic care according to claim 14 or 15, characterized in that it is intended for dermal filling, and/or subcutaneous filling, and/or filling of muscle, and/or of cartilage, and/or of the mucosae.
